(19) United States
(12) Patent Application Publication
(10) Pub. No.: US 2008/0015250 A1
(43) Pub. Date: Jan. 17, 2008

(54) Ultraviolet absorbing ophthalmic compositions

(76) Inventors: Mitchell H. Friedlaender, La Jolla, CA (US); Harun Takruri, Newport Beach, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/819,492

(22) Filed: Jun. 27, 2007

Related U.S. Application Data
(60) Provisional application No. 60/826,475, filed on Sep. 21, 2006.
(60) Provisional application No. 60/805,973, filed on Jun. 27, 2006.

(51) Int. Cl. A61K 31/355; A61K 31/34; A61P 27/02

(52) U.S. Cl. 514/458; 514/474

(57) ABSTRACT

The present invention is directed to a composition comprising: (a) one or more UV-absorbing agents, wherein all the UV-absorbing agents are in solution; and (b) an excipient; wherein the composition is ophthalmically acceptable, and wherein the composition does not contain an active agent. In some embodiments, the UV-absorbing agent is a vitamin E compound, ascorbic acid, sodium ascorbate, sorbic acid, potassium sorbate, amino acid, or combination thereof. The present invention is also directed to kits comprising the composition described herein.

This application claims the benefit of the filing date of U.S. Appl. No. 60/805,973, filed Jun. 27, 2006, and U.S. Appl. No. 60/826,475, filed Sep. 21, 2006, the entirety of both which are fully incorporated by reference herein.

### BACKGROUND OF THE INVENTION

1. Field of the Invention

The present invention is directed to ophthalmic compositions. More particularly, the present invention is directed to a composition comprising: (a) one or more UV-absorbing agents, wherein all the UV-absorbing agents are in solution; and (b) an excipient; wherein the composition is ophthalmically acceptable, and wherein the composition does not contain an active agent. In some embodiments, the UV-absorbing agent is a vitamin E compound, ascorbic acid, sodium ascorbate, sorbic acid, potassium sorbate, amino acid, or combination thereof. The present invention is also directed to kits comprising the composition described herein.

2. Background Art

Normally, tear producing glands supply moisture and lubrication to the eye. However, sometimes the levels of naturally produced moisture and lubricants on the eye are reduced, leading to a condition termed “dry eye.” Conditions which may cause dry-eye include aging, autoimmunce diseases such as rheumatoid arthritis and lupus, injury, laser vision correction, and environmental factors. Some medications can also cause dry eyes, e.g. tricyclic antidepressants, decongestants, antihistamines, diuretics, and some medicines for Parkinson's disease. Symptoms of dry eye include the sensation of dryness, itching, burning, stinging, irritation, redness, excessive tearing, blurred vision and discomfort with reading, computer use, or watching TV. Regardless of the cause of dry-eye, ophthalmic solutions, referred to as “artificial tears”, and ointments or gels, referred to as “lubricants” are often used for treatment. These artificial tears and lubricants attempt to alleviate discomfort by restoring moisture and lubrication.

Artificial tears commonly use demulcents to soothe or protect the ocular surface. While demulcents are effective at reducing mechanical friction, they generally do not protect the eye against ultraviolet radiation. A need exists in the art for an ophthalmic solution that comprises an ultraviolet absorbing agent.

### BRIEF SUMMARY OF THE INVENTION

The present invention is related to a composition comprising: (a) one or more UV-absorbing agents, wherein all the UV-absorbing agents are in solution; and (b) an excipient; wherein the composition is ophthalmically acceptable, and wherein the composition does not contain an active agent.

In some embodiments, the UV-absorbing agent is a vitamin E compound, ascorbic acid, sodium ascorbate, sorbic acid, potassium sorbate, amino acid, or combination thereof. Vitamin E compounds can include tocopheryl acetate, tocopheryl acetate succinate, tocophersolan, and combinations thereof. In some embodiments, the vitamin E compound is about 0.01% to about 10% w/v of the composition.

In some embodiments, the UV-absorbing agent is ascorbic acid, sodium ascorbate, or combination thereof. The ascorbic acid, sodium ascorbate, or combination thereof can be about 0.01% to about 6.0% w/v of the composition.

In some embodiments, the UV-absorbing agent is sorbic acid, potassium sorbate, or combination thereof. The sorbic acid, potassium sorbate, or combination thereof can be about 0.01% to about 2% w/v of the composition.

In the present invention, the excipient can comprise a demulcent, antimicrobial agent, tonicity agent, buffering agent, stabilizing agent, or combination thereof. The demulcent can be selected from a cellulose derivative, dextran, gelatin, polyol, polyvinyl alcohol, povidone, chondroitin sulfate, hyaluronic acid, and combinations thereof. The cellulose derivative can be selected from the group consisting of carboxymethylcellulose, one or more salts of carboxymethylcellulose, hydroxyethyl cellulose, hypromellose, methylcellulose, and combinations thereof. The polyol can be selected from the group consisting of glycerin, polyethylene glycol, polysorbate, propylene glycol, and combinations thereof. In some embodiments, the antimicrobial agent is selected from the group consisting of edetate disodium, benzalkonium chloride, and combinations thereof. In some embodiments, the tonicity agent is selected from the group consisting of sodium chloride, potassium chloride, calcium chloride, and combinations thereof. In some embodiments, the buffering agent is selected from the group consisting of citric acid, sodium citrate, boric acid, sodium borate, one or more sodium salts of phosphoric acid, one or more potassium salts of phosphoric acid, sodium bicarbonate, and combinations thereof.

In some embodiments, the composition of the present invention does not contain Vitamin A or a Vitamin A derivative.

The present invention is also directed to a composition comprising: (a) one or more UV-absorbing agents, wherein all the UV-absorbing agents are in solution; and (b) an excipient; wherein the composition is ophthalmically acceptable, and wherein the composition does not contain an active agent.

The present invention is also related to a composition comprising: (a) a cellulose derivative; (b) a vitamin E compound in solution; (c) an antimicrobial; and (d) a buffer, wherein the composition is ophthalmically acceptable.

The present invention is also related to a composition comprising: (a) a cellulose derivative; (b) tocopheryl acetate and tocophersolan in solution; (c) an antimicrobial; and (d) a buffer; wherein the composition is ophthalmically acceptable.

The present invention is also related to a composition comprising: (a) carboxymethylcellulose sodium; (b) tocophersolan; (c) tocopheryl acetate; (d) potassium sorbate; (e) ascorbic acid; (f) benzalkonium chloride; (g) propylene glycol; (h) sodium hydroxide/hydrochloric acid; and (i) water, wherein the composition has a pH of about 7.0.

The present invention is also related to a composition comprising: (a) carboxymethylcellulose sodium; (b) tocophersolan; (c) tocopheryl acetate; (d) potassium sorbate; (e) ascorbic acid; (f) sodium borate; (g) edetate disodium; (h) benzalkonium chloride; (i) propylene glycol; (j) sodium hydroxide/hydrochloric acid; and (k) water, wherein the composition has a pH of about 6.5.

The present invention is also related to a composition comprising: (a) hypromellose; (b) tocophersolan; (c) tocopheryl acetate; (d) potassium sorbate; (e) ascorbic acid; (f) sodium borate; (g) edetate disodium; (h) benzalkonium chloride; (i) glycerin; (j) sodium hydroxide/hydrochloric acid; and (k) water, wherein the composition has a pH of about 6.5.

The present invention is also related to a kit comprising: (a) the composition of the present invention; and (b) instructions for using the composition of (a). In some embodiments, the invention is related to a kit comprising: (a) the composition of the present invention; wherein the composition is individually packaged for a single administration; and (b) instructions for using the composition of (a).

### BRIEF DESCRIPTION OF THE DRAWINGS/FIGURES

FIG. 1: The UV radiation absorbing profile of the composition of Example 1 (C.) was compared to that of two commercial compositions, Refresh Tears® lubricant eye drops (B.) (ALLERGAN, Irvine, Calif.) and Thera Tears® (A.) (ADVANCED VISION RESEARCH, Woburn, Mass.). FIG. 1 reflects the absorption of radiation through a 1 cm path length for each of these compositions as tested by a Beckman DU-7400 UV-vis spectrophotometer (BECKMAN COULTER, Fullerton, Calif.). The Y-axis represents the % transmission, the X-axis represents the wavelength (in nm).

### DETAILED DESCRIPTION OF THE INVENTION

The present invention is directed to a composition comprising: (a) one or more UV-absorbing agents, wherein all the UV-absorbing agents are in solution; and (b) an excipient, wherein the composition is ophthalmically acceptable. The present invention is also directed to a composition comprising (a) one or more UV-absorbing agents, wherein all the UV-absorbing agents are in solution; and (b) an excipient; wherein the composition is ophthalmically acceptable, and wherein the composition does not contain an active agent.

The present invention is also directed to a composition comprising: (a) one or more UV-absorbing agents, wherein all the UV-absorbing agents are in solution; and (b) an excipient; wherein the composition is ophthalmically acceptable, and wherein the composition does not contain an active agent.

It is to be noted that the term “a” or “an” entity refers to one or more of that entity; for example, “a UV-absorbing agent,” is understood to represent one or more UV-absorbing agents. As such, the terms “a” (or “an”), “one or more,” and “at least one” can be used interchangeably herein.

The term “UV-absorbing agent” refers to a compound or composition that inherently absorbs radiation in the ultraviolet wavelengths. Ultraviolet radiation is sometimes referred to as ultraviolet light. Ultraviolet radiation or ultraviolet wavelengths include UV-A radiation (325 nm-390 nm), UV-B radiation (295 nm-325 nm) and UV-C radiation (200 nm-295 nm). The percentage of radiation absorbed in the UV region can vary depending on the wavelength of radiation. As defined herein, a UV-absorbing agent must absorb radiation substantially throughout the UV wavelength region, i.e., from 200 nm to 390 nm. In other words, a UV-absorbing agent of the present invention absorbs radiation in UV-A, UV-B and UV-C wavelengths. In some embodiments, the UV-absorbing agent absorbs radiation from 200 nm to 390 nm, or from 210 nm to 390 nm, from 220 nm to 390 nm. In some embodiments, the UV-absorbing agent absorbs radiation from 200 nm to 380 nm, from 200 nm to 370 nm, or from 200 nm to 360 nm. In some embodiments, the UV-absorbing agent absorbs radiation from 220 nm to 370 nm. In some embodiments, the UV-absorbing agent transmits less than about 90%, less than about 50%, less than about 40%, less than about 20%, less than about 10%, less than about 5% or less than about 3% of the radiation at 200 nm. In some embodiments, the UV-absorbing agent transmits less than about 90%, less than about 50%, less than about 40%, less than about 20%, less than about 10%, less than about 5% or less than about 3% of the radiation at 295 nm. In some embodiments, the UV-absorbing agent transmits less than about 90%, less than about 50%, less than about 40%, less than about 20%, less than about 10%, less than about 5% or less than about 3% of the radiation at 300 nm. In some embodiments, the UV-absorbing agent transmits less than about 90%, less than about 50%, less than about 40%, less than about 20%, less than about 10%, less than about 5% or less than about 3% of the radiation at 325 nm. In some embodiments, the UV-absorbing agent transmits less than about 90%, less than about 50%, less than about 40%, less than about 20%, less than about 10%, less than about 5% or less than about 3% of the radiation at 390 nm. One of skill in the art will realize that the term “UV-absorbing agent” is not limited to agents that only absorb radiation in the UV region. Thus, UV agents are capable of absorbing radiation at other wavelengths in addition to UV wavelengths.

The extent of absorption at a given wavelength can vary depending on various factors, e.g., the nature of the UV-absorbing compound/composition, concentration of the UV-absorbing compound/composition, the pathlength of the sample cell, and intensity of the radiation. A compound is considered to “absorb,” as defined herein, if the transmitted radiation is less than 80% of the incident radiation. Transmittance of UV radiation can be determined by various methods known to those in the art, such as, but not limited to, spectrophotometry. In some embodiments, transmittance is determined using a Beckman DU-7400 UV-vis spectrophotometer (BECKMAN COULTER, Fullerton, Calif.).

Various UV-absorbing agents can be used. In some embodiments, the UV-absorbing agent is a vitamin E compound, ascorbic acid, sodium ascorbate, sorbic acid, potassium sorbate, amino acid, or combination thereof. In some embodiments, the UV-absorbing agent is vitamin E compound, ascorbic acid, sodium ascorbate, sorbic acid, potassium sorbate. The term “vitamin E compounds” refers to vitamin E and soluble salts and esters thereof. For example, vitamin E compounds can include tocopheryl acetate, tocopheryl acetate succinate, tocophersolan, and combinations thereof. The term tocophersolan refers to a vitamin E polyethylene glycol succinate, e.g., d-alpha tocopheryl polyethylene glycol 1000 succinate (Eastman, Kingsport, Tenn.). Polyethylene glycol derivatives of the present invention can be of various molecular weights. In some embodiments, the UV-absorbing agent is a mixture of tocopheryl acetate and tocophersolan. In some embodiments, the UV-absorbing agent is a mixture of tocopheryl acetate, tocophersolan, and ascorbic acid.

The vitamin E compound can be present in various concentrations. For example, the vitamin E compound can be about 0.005% to about 20% w/v of the composition, about 0.01% to about 10.0% w/v of the composition, about 0.01% to about 5.0% w/v of the composition, or about 0.05% to about 2% w/v of the composition.

In some embodiments, the UV-absorbing agent is ascorbic acid (vitamin C), sodium ascorbate, combinations thereof, or other salts or esters thereof. The ascorbic acid, sodium ascorbate, or combination thereof can be present in various concentrations. For example, ascorbic acid, sodium ascorbate, or combination thereof can be about 0.005% to about 10.0% w/v of the composition, about 0.01% to about 6.0% w/v of the composition, about 0.05% to about 4.0% w/v of the composition, about 0.1% to about 2.0% w/v of the composition, or about 0.5% to about 1.5% w/v of the composition.

In some embodiments, the composition of the present invention does not contain Vitamin A or a Vitamin A derivative. Vitamin A and various Vitamin A derivatives are not generally considered to be water soluble, thus in some embodiments are not suitable for the present invention. Vitamin A refers to retinol. Vitamin A derivatives include, but are not limited to, esters of retinol such as vitamin A acetate, vitamin A palmitate and the like, retinoic acid and retinoic esters such as retinoic acid methyl ester and the like.

In some embodiments, the UV-absorbing agent is sorbic acid, potassium sorbate, or combination thereof, or other salts or esters thereof. The sorbic acid, potassium sorbate, or combination thereof can be present in various concentrations. For example, the sorbic acid, potassium sorbate, or combination thereof can be about 0.005% to about 8.0% w/v of the composition, about 0.005% to about 4.0% w/v of the composition, about 0.01% to about 2.0% w/v of the composition, or about 0.1% to about 1.0% w/v of the composition.

The compositions of the present inventions are “ophthalmically acceptable.” The term “ophthalmically acceptable” refers to those compounds, materials, compositions, and/or solutions which are, within the scope of sound medical judgment, suitable specifically for contact with the tissues of the eye, and the area surrounding the eye without excessive toxicity, irritation, allergic response, or other problem complications commensurate with a reasonable benefit/risk ratio.

In the present invention, the ophthalmically acceptable excipient can comprise a demulcent, antimicrobial agent, tonicity agent, buffering agent, stabilizing agent, or combination thereof.

Various demulcents can be used. The term “demulcent” refers to any compound or composition that when applied to an ocular area can lubricate, soothe and/or protect the mucous membrane of the eye. In some embodiments, the demulcent is selected from a cellulose derivative, dextran, gelatin, polyol, polyvinyl alcohol, povidone, chondroitin sulfate, hyaluronic acid, and combinations thereof. Various cellulose derivatives can be used. Examples include, but are not limited to, carboxymethylcellulose, one or more salts of carboxymethylcellulose, hydroxyethyl cellulose, hypromellose, methylcellulose, and combinations thereof. The term “polyol” refers to a compound with greater than 2 alcohol groups. Examples of polyols include, but are not limited to glycerin, polyethylene glycol, polysorbate, propylene glycol, and combinations thereof. Various concentrations of polyols can be used in the present invention. In some embodiments, the polyol is about 0.01% to about 20.0% w/v of the composition, about 0.1% to about 10.0% w/v of the composition, or about 0.5% to about 5.0% w/v of the composition.

The present invention is directed to compositions comprising UV-absorbing agents in solution. An agent “in solution” refers to an agent in a composition wherein the agent is completely dissolved at room temperature, resulting in an essentially homogeneous dispersion of that agent in the composition. One of skill in the art will recognize that only a certain amount of UV-absorbing agent can be dissolved in a set amount of solvent. Thus, in accordance with the present invention, the concentration of the UV-absorbing agent must remain below the solubility constant for the given UV-absorbing agent in the composition. For example, in some embodiments, UV-absorbing agents refer to agents with a solubility in the composition at 23° C. of at least 0.1 g/L, at least 1.0 g/L, at least 10 g/L, at least 20 g/L, at least 50 g/L, at least 75 g/L, at least 90 g/L, or at least 100 g/L. It will be understood by one of skill in the art that compounds with low solubility in an aqueous environment may increase their solubility in the aqueous environment by adding a co-solvent. Use of co-solvents can increase the solubility by several orders of magnitude. For example, the solubility of tocopheryl acetate can be increased by the addition of tocophersolan. In some embodiments, co-solvents can be selected from the group consisting of propylene glycol, polyethylene glycols, sorbitol, and combinations thereof. In some embodiments, the solvent is substantially water, although one of skill in the art will recognize that other components of the composition of the invention can be present in the water and may be considered to be “solvents” or “co-solvents” along with water.

In some embodiments, surfactants or complexing agents can be used to increase the solubility of a UV-absorbing agent in the composition of the present invention. A surfactant or surface active agent is amphipathic, meaning it has a polar end (the circular head) and a nonpolar end (the tail). Generally, a surfactant forms micelles when placed in an aqueous environment. In some embodiments, the composition of the present invention does not contain a surfactant. However, in other embodiments, the composition of the present invention further comprises a surfactant. In some embodiments, the composition comprises a complexing agent. Complexing agents rely on relatively weak forces such as London forces, hydrogen bonding and hydrophobic interactions to increase the solubility of a solute. Examples of complexing agents include, but are not limited to inorganic compounds (e.g., I_{B}), coordination compounds (e.g., hexaminecobalt(III) chloride), chelates (e.g., EDTA, EGTA), metal-olefin compounds (e.g., ferrocene), and inclusion compounds (e.g., choleic acid, cyclodextrins). In some embodiments, the composition of the present invention does not contain a complexing agent.

While not being bound by any theories, in some embodiments, compositions comprising UV-agents in solution provide increased lubrication properties and/or decreased irritability compared to compositions comprising UV-absorbing agents not in solution. Additionally, compositions comprising UV-absorbing agents in solution can be easier to handle (e.g., thus providing benefits during manufacturing) and can provide a more uniform distribution of the UV-agent present in the composition of the present invention relative to compositions in which the UV-absorbing agent is not in solution.

In some embodiments of the present invention, the composition does not comprise an active agent. The term “active agent”, as used herein refers to a chemical compound, macromolecule, or composition of matter which, when administered to an organism (human or animal subject) induces a desired pharmacologic and/or physiologic effect by local and/or systemic action. The term therefore encompasses those compounds or chemicals traditionally regarded as drugs or biopharmaceuticals (including molecules such as peptides, proteins, nucleic acids). Examples of active agents include antibiotics and antiviral agents; analgesics and analgesic combinations, antiseptics, antihistamines; anti-inflammatory agents, hormones or steroids, vasodilators; proteins, peptides, and fragments thereof (whether naturally occurring, chemically synthesized or recombinantly produced); and nucleic acid molecules.

In some embodiments of the present invention the composition comprises an antimicrobial agent. In some embodiments, the antimicrobial agent is selected from the group consisting of edetate disodium, benzalkonium chloride, and combinations thereof. Various concentrations of antimicrobial agent can be used, dependant on, e.g., the type of antimicrobial agent, its mechanism of action, and/or its ophthalmic acceptability. In some embodiments, the antimicrobial agent is about 0.00001% to about 4.0% w/v of the composition, or about 0.0001% to about 1.0% w/v of the composition, or about 0.001% to about 0.1% w/v of the composition.

Various tonicity agents can be used. Tonicity agents can be used to adjust the salt concentration of the composition, provided the agent is ophthalmically acceptable. In some embodiments, the tonicity agent is selected from the group consisting of dextrose, sodium chloride, potassium chloride, calcium chloride, and combinations thereof. In some embodiments, the tonicity agent is used to produce an isotonic composition. In some embodiments, the tonicity agent is used to produce a hypotonic composition.

Additional excipients can include viscosity agents. Viscosity agents can be used to adjust the coefficient of viscosity for the composition of the present invention. In some embodiments, a viscosity agent is the same as a demulcent. Viscosity agents useful in the compositions of the present invention can include, but are not limited to, carbopol, cellulose derivatives such as hydroxypropylmethyl cellulose, sodium carboxymethyl cellulose, hydroxyethyl cellulose, other viscosity inducing materials useful in ophthalmic formulations and the like.

The composition of the present invention can also include an acid, base or buffer. Acid/bases/buffers can be included to provide and/or maintain the present compositions at a pH in the physiologically acceptable range, more preferably in a range of about 3 to about 9, or about 4 to about 8.5, still more preferably about 5 to about 8.5 or about 5.5 to about 8.0, and especially about 6.0 to about 8.0 or about 6.5 to about 7.0. As one of skill in the art will recognize, the pH can vary over time, depending on various factors, e.g., stability of the composition, amount of exposure to the atmosphere, strength of buffer, etc. Thus, as used herein, when referring to compositions, kits or methods of the present invention, any specified pH refers to the pH at any time between the time of manufacturing and time of administering.

The term “buffer” refers to an ophthalmically acceptable compound or composition that is capable of neutralizing both acids and bases and thereby maintaining the original acidity or basicity of the composition. Buffers can include, but are not limited to, phosphate buffers (e.g., sodium and potassium phosphates), phosphates, bicarbonate, citrate, borate, acetate buffers, citrate buffers, tromethamine buffers and combinations thereof. Preferred buffers are selected from the group consisting of citric acid, sodium citrate, boric acid, sodium borate, one or more sodium salts of phosphoric acid, one or more potassium salts of phosphoric acid, sodium bicarbonate, and combinations thereof.

Acids useful in the present compositions can include boric acid, hydrochloric acid, acetic acid, other acids which are ophthalmically acceptable in the concentrations used, and the like.

Bases which can be included in the present compositions include, but are not limited to, sodium and/or potassium hydroxides, other alkali and/or alkaline earth metal hydroxides, organic bases, other bases which are ophthalmically acceptable in the concentrations used, and the like.

The present compositions can be in any physical form suitable to be administered to the eye. Such physical forms include, but are not limited to, liquids (e.g., solutions), semi-solids (gels, creams, ointments, etc.), and the like. Each of these physical forms of the present compositions can be prepared using techniques and processing which are conventional and well known in the art. For a more detailed discussion of the preparation and administration of ophthalmic formulations see Remington's Pharmaceutical Sciences, 15 Ed., Pgs. 1489 to 1504 (1975) which is incorporated in its entirety herein by reference.

The composition of the present invention can be administered to the eye for treatment of various conditions. In some embodiments, the composition is administered for the treatment of dry eye (keratitis sicca). Thus, in some embodiments, the present invention is directed to a method of treating dry eye, the method comprising administration of the composition of the present invention to the eye. Due to the presence of the UV-absorbing agent, the composition of the present invention can be used to reduce the exposure of the eye to UV radiation. In some embodiments, the present invention is directed to a method of reducing exposure of an eye to UV radiation, the method comprising applying the composition of the present invention to the eye. In some embodiments, the present invention is directed to a method of reducing the damaging effects of UV radiation, the method comprising applying the composition of the present invention to the eye. UV radiation has been implicated in the development of pterygia, corneal degenerations, cataract, macular degeneration, and solar burns of the cornea and retina. Thus, the present invention is directed to a method of preventing of 1) reducing the frequency of occurrence of, 2) reducing the symptoms or ill effects of, 3) preventing or 4) otherwise interfering with the above conditions.

The present invention is related to a composition comprising: (a) one or more vitamin E compounds in solution; and (b) an excipient, wherein the composition is ophthalmically acceptable, and wherein the composition does not contain an active agent.

The present invention is also related to a composition comprising: (a) a cellulose derivative; (b) a vitamin E compound in solution; (c) an antimicrobial; and (d) a buffer, wherein the composition is ophthalmically acceptable.

The present invention is also related to a composition comprising: (a) a cellulose derivative; (b) tocopheryl acetate and tocophersolan in solution; (c) an antimicrobial; and (d) a buffer; wherein the composition is ophthalmically acceptable.

The present invention is also related to a composition comprising: (a) carboxymethylcellulose sodium; (b) tocophersolan; (c) tocopheryl acetate; (d) potassium sorbate; (e) ascorbic acid; (f) benzalkonium chloride; (g) propylene glycol; (h) sodium hydroxide/hydrochloric acid; and (i) water, wherein the composition has a pH of about 7.0.

The present invention is also related to a composition comprising: (a) carboxymethylcellulose sodium; (b) tocophersolan; (c) tocopheryl acetate; (d) potassium sorbate; (e) ascorbic acid; (f) sodium borate; (g) edetate disodium; (h) benzalkonium chloride; (i) propylene glycol; (j) sodium hydroxide/hydrochloric acid; and (k) water, wherein the composition has a pH of about 6.5.

The present invention is also related to a composition comprising: (a) hypromellose; (b) tocophersolan; (c) tocopheryl acetate; (d) potassium sorbate; (e) ascorbic acid; (f) sodium borate; (g) edetate disodium; (h) benzalkonium chloride; (i) glycerin; (j) sodium hydroxide/hydrochloric acid; and (k) water, wherein the composition has a pH of about 6.5.

The present invention is also related to a kit comprising: (a) the composition of the present invention; and (b) instructions for using the composition of (a). In some embodiments, the invention is related to a kit comprising: (a) the composition of the present invention, wherein the composition is individually packaged for a single administration; and (b) instructions for using the composition of (a).

The composition can be individually packaged, e.g., in a bottle, jar, ampoule, tube, syringe, envelope, container, or vial. Alternatively, the composition can be contained in a package that is capable of holding multiple units, e.g., in resealable glass or plastic packages. In some kits, the components of the composition are mixed together immediately preceding their usage. For example, in some embodiments one or more dry components of the composition of the kit are packaged in a separate container, e.g., a plastic bottle, and then mixed with one or more of the liquid components of the composition immediately prior to use. Optionally, the kit of the present invention can include a dropper or other device for transferring/administering the composition to a subject.

Optionally, the kit can further comprise printed matter containing instructions for using the composition of the present invention. For example, such printed instructions can be in a form prescribed by a governmental agency regulating the manufacture, use or sale of pharmaceuticals or biological products, which notice reflects approval by the agency of the manufacture, use or sale for human application. In some embodiments, the kit further comprises printed matter, which, e.g., provides information on the use of the composition or a pre-recorded media device which, e.g., provides information on the use of the method of the present invention.

“Printed matter” can be, for example, a book, booklet, brochure, leaflet or the like. The printed matter can describe the use of the composition of the present invention. Possible formats include, but are not limited to, a bullet point list, a list of frequently asked questions (FAQ) or a chart. Additionally, the information to be imparted can be illustrated in non-textual terms using pictures, graphics or other symbols.

The kit can also include a container for storing the components of the kit. The container can be, for example, a bag, box, envelope or any other container that would be suitable for use in the present invention. In some embodiments, the container is large enough to accommodate each component of the present invention. However, in some cases, it can be desirable to have a smaller container which is large enough to carry only some of the components of the present invention.

### EXAMPLES

### Example 1

An ophthalmic solution was made as described in Table 1:

TABLE 1

| Ingredient | Percent, w/v |
|---|---|
| Carboxymethylcellulose Sodium | 0.4 |
| Tocophersolan | 0.4 |
| Tocopheryl Acetate | 0.1 |
| Potassium Sorbate | 0.1 |
| Ascorbic Acid | 0.1 |
| Benzalkonium Chloride | 0.005 |
| Propylene Glycol | 1.5 |
| Sodium Hydroxide/Hydrochloric Acid | adjust pH to 7.0 |
| Purified Water | q.s. 100 |

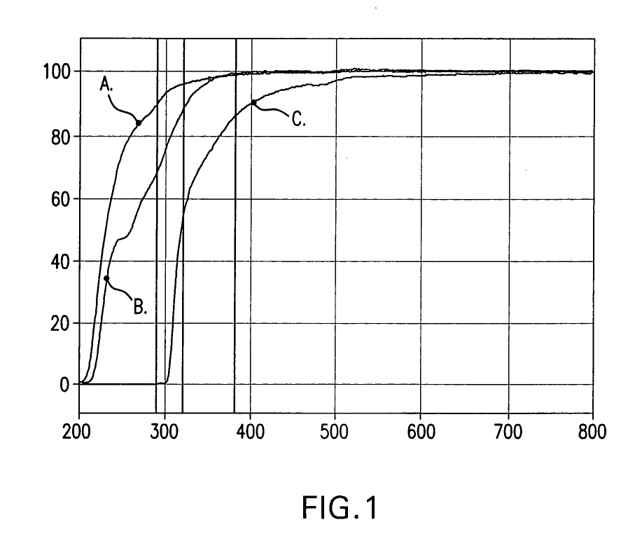

The UV radiation absorbing profile of the composition of Table 1 was compared to that of two commercial compositions, Refresh Tears® lubricant eye drops (ALLERGAN, Irvine, Calif.) and Thera Tears® (ADVANCED VISION RESEARCH, Woburn, Mass.). FIG. 1 demonstrates the absorption of radiation through a 1 cm path length for each of these compositions using a Beckman DU-7400 UV-vis spectrophotometer (BECKMAN COULTER, Fullerton, Calif.).

### Example 2

An ophthalmic solution was made as described in Table 2:

TABLE 2

| Ingredient | Percent, w/v |
|---|---|
| Carboxymethylcellulose Sodium | 0.4 |
| Tocophersolan | 0.4 |
| Tocopheryl Acetate | 0.1 |
| Potassium Sorbate | 0.1 |
| Ascorbic Acid | 0.1 |
| Sodium Borate | 0.1 |
| Edetate Disodium | 0.01 |
| Benzalkonium Chloride | 0.005 |
| Propylene Glycol | 1.5 |
| Sodium Hydroxide/Hydrochloric Acid | adjust pH to 6.5 |
| Purified Water | q.s. 100 |

### Example 3

An ophthalmic solution can be made as described in Table 3:

TABLE 3

| Ingredient | Percent, w/v |
|---|---|
| Hypromellose | 1.0 |
| Tocophersolan | 2.0 |
| Tocopheryl Acetate | 0.1 |
| Potassium Sorbate | 0.5 |
| Ascorbic Acid | 1.0 |
| Sodium Borate | 1.0 |
| Edetate Disodium | 0.01 |
| Benzalkonium Chloride | 0.005 |
| Glycerin | 1.0 |
| Sodium Hydroxide/Hydrochloric Acid | adjust pH to 6.5 |
| Purified Water | q.s. 100 |

It is to be appreciated that the Detailed Description section, and not the Summary and Abstract sections, is intended to be used to interpret the claims. These examples illustrate possible compositions used in the present invention. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A composition comprising:
(a) one or more UV-absorbing agents, wherein all the UV-absorbing agents are in solution; and
(b) an excipient;
wherein the composition is ophthalmically acceptable, and wherein the composition does not contain an active agent.

2. The composition of claim 1, wherein the UV-absorbing agent is a vitamin E compound, ascorbic acid, sodium ascorbate, sorbic acid, potassium sorbate, an amino acid, or combination thereof.

3. The composition of claim 2, wherein the UV-absorbing agent is a vitamin E compound, wherein the vitamin E compound is selected from the group consisting of tocopheryl acetate, tocopheryl acetate succinate, tocophersolan, and combinations thereof.

4. The composition of claim 3, wherein the UV-absorbing agent is a mixture of tocopheryl acetate and tocophersolan.

5. The composition of claim 3, wherein the vitamin E compound is about 0.01% to about 10.0% w/v of the composition.

6. The composition of claim 2, wherein the UV-absorbing agent is ascorbic acid, sodium ascorbate, or combinations thereof.

7. The composition of claim 6, wherein the ascorbic acid, sodium ascorbate, or combination thereof is about 0.01% to about 6.0% w/v of the composition.

8. The composition of claim 2, wherein the UV-absorbing agent is sorbic acid, potassium sorbate, or combinations thereof.

9. The composition of claim 8, wherein the sorbic acid, potassium sorbate, or combination thereof is about 0.01% to about 2.0% w/v of the composition.

10. The composition of claim 1, wherein the excipient comprises a demulcent, antimicrobial agent, tonicity agent, buffering agent, stabilizing agent, or combinations thereof.

11. The composition of claim 10, wherein the demulcent is selected from a cellulose derivative, dextran, gelatin, polyol, polyvinyl alcohol, povidone, chondroitin sulfate, hyaluronic acid, and combinations thereof.

12. The composition of claim 11, wherein the cellulose derivative is selected from the group consisting of carboxymethylcellulose, one or more salts of carboxymethylcellulose, hydroxyethyl cellulose, hypromellose, methylcellulose, and combinations thereof.

13. The composition of claim 11, wherein the polyol is selected from the group consisting of glycerin, polyethylene glycol, polysorbate, propylene glycol, and combinations thereof.

14. The composition of claim 10, wherein the antimicrobial agent is selected from the group consisting of edetate disodium, benzalkonium chloride, and combinations thereof.

15. The composition of claim 10, wherein the tonicity agent is selected from the group consisting of sodium chloride, potassium chloride, calcium chloride, and combinations thereof.

16. The composition of claim 10, wherein the buffering agent is selected from the group consisting of citric acid, sodium citrate, boric acid, sodium borate, one or more sodium salts of phosphoric acid, one or more potassium salts of phosphoric acid, sodium bicarbonate, and combinations thereof.

17. The composition of claim 1, wherein the composition does not contain Vitamin A or a Vitamin A derivative.

18. A composition comprising:
(a) a cellulose derivative;
(b) a vitamin E compound in solution;
(c) an antimicrobial; and
(d) a buffer;
wherein the composition is ophthalmically acceptable.

19. A composition comprising:
(a) a cellulose derivative;
(b) tocopheryl acetate and tocophersolan in solution;
(c) an antimicrobial; and
(d) a buffer;
wherein the composition is ophthalmically acceptable.

20. (canceled)

21. (canceled)

22. (canceled)

23. A kit comprising:
(a) the composition of claim 1; and
(b) instructions for using the composition of (a).

24. A kit comprising:
(a) the composition of claim 1, wherein the composition is individually packaged for a single administration; and
(b) instructions for using the composition of (a).
